(19) United States
(12) Patent Application Publication
(10) Pub. No.: US 2021/0236129 A1
(43) Pub. Date: Aug. 5, 2021

(54) LUMINAL APPOSITION DEVICES

(71) Applicant: Mayo Foundation for Medical Education and Research, Rochester, MN (US)

(72) Inventor: Mark D. Topazian, Rochester, MN (US)

(21) Appl. No.: 17/054,295

(22) PCT Filed: May 10, 2019
(86) PCT No.: PCT/US2019/031768

§ 371 Date: Nov. 10, 2020

Related U.S. Application Data
(60) Provisional application No. 62/670,294, filed on May 11, 2018.

(51) Int. Cl. A61B 17/11; A61B 17/34

(52) U.S. Cl. CPC A61B 17/11; A61B 2017/00867; A61B 17/3468

(57) ABSTRACT

A device for holding two anatomical structures in apposition. The device includes a first holding member, a second holding member, and a connecting member extending between the first holding member and the second holding member.

### CROSS-REFERENCE TO RELATED APPLICATIONS

This application claims priority to U.S. Application Ser. No. 62/670,294, filed on May 11, 2018. The disclosure of the prior application is considered part of the disclosure of this application, and is incorporated in its entirety into this application.

### BACKGROUND

### Technical Field

This document relates to methods and materials for holding two anatomic structures in apposition. For example, this document relates to methods and devices for holding a stomach and jejunum in apposition.

### Background Information

A gastrojejunostomy is a surgical procedure that directly connects the stomach to the small intestine, specifically the jejunum. The jejunum is the second part of the small intestine, located after the duodenum. The stomach and jejunum can be connected by a small tube that allows food, liquids, and/or medications to bypass the duodenum. A gastrojejunostomy can be beneficial to individuals who cannot properly digest food, such as those who have stomach or duodenum blockages. These blockages can be caused by stomach or pancreatic cancer.

### SUMMARY

This document describes methods and materials for holding two anatomic structures in apposition. For example, this document describes methods and devices for holding a stomach and jejunum in apposition. This disclosure is directed to coil tags with an intervening segment connecting the coils for holding the lumens of two anatomical structures in apposition.

In one aspect, this disclosure is directed to a device for holding two anatomic structures in apposition. The device includes a first holding member, a second holding member, and a connecting member extending between the first holding member and the second holding member.

In some cases, the device can be made of shape memory material. In some cases, the connecting member can be substantially straight extending between the first holding member and the second holding member. In some cases, the connecting member can be curved such that the first holding member and the second member are in proximity to one another. In some cases, the connecting member can be non-linear. In some cases, the connecting member can be configured to anchor the device in a tissue. In some cases, the first holding member and the second holding member can extend perpendicular from the connecting member in a radially expanding spiral pattern. In some cases, the first holding member and the second holding member can extend longitudinally from the connecting member in a helical configuration. In some cases, the first holding member and the second holding member can extend from the connecting member in a radially expanding conical configuration. In some cases, the first holding member and the second hold member can extend toward the connecting member in a radially expanding conical configuration. In some cases, the connecting member can have a helical configuration. In some cases, the connecting member can include a helical portion. In some cases, the first holding member can include a distal protrusion. In some cases, the device can further include a hinge coupling the first holding member and the connecting member.

In some cases, the first holding member can configured to be positioned in a first anatomical structure, the second holding member can be configured to be positioned in the first anatomical structure, and the connecting member can configured to extend through the first anatomical structure and a second anatomical structure. In some cases, the first holding member can be configured to be positioned in a first anatomical structure, the second holding member can be configured to be position in a second anatomical structure, and the connecting member can be configured to extend through the first anatomical structure and the second anatomical structure. In some cases, the first holding member can be configured to allow passage of a stent through a portion of the first holding member. In some cases, the second holding member can be configured to allow passage of a stent through a portion of the second holding member. In some cases, the device can further include a second connecting member configured to extend between the first holding member and the second holding member opposite the connecting member.

In one aspect, this disclosure is directed to a method of holding two anatomic structures in apposition. The method includes implanting a device in the two anatomic structures by ejecting a first holding member from a needle in a first anatomical structure, withdrawing the needle into a second anatomical structure, and ejecting a second holding member from the needle in the second anatomical structure.

In some cases, withdrawing the needle can include ejecting a connecting member in the first anatomical structure and the second anatomical structure, where the connecting member extends between the first holding member and the second holding member. In some cases, the method can include implanting a plurality of devices. In some cases, the plurality of devices can be two to six devices. In some cases, the method can include implanting a stent. In some cases, implanting the stent can include implanting the stent in a first loop of the first holding member. In some cases, implanting the stent can include implanting the stent in a second loop of the second holding member. In some cases, implanting the stent can include implanting the stent between the plurality of devices.

Particular embodiments of the subject matter described in this document can be implemented to realize one or more of the following advantages. The coiled tags described herein can be implanted using a needle. Accordingly, the devices and methods provided herein can increase the safety and efficiency of connecting two anatomical structures. Further, the devices and methods can reduce the risk of the procedure failing due to anatomic structures movement.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods and examples are illustrative only and not intended to be limiting.

The details of one or more embodiments of the invention are set forth in the accompanying drawings and the description below. Other features, objects, and advantages of the invention will be apparent from the description, drawings, and claims.

### DESCRIPTION OF DRAWINGS

FIG. 1 is a perspective view of a first end of a coiled tag, in accordance with some embodiments provided herein.

FIGS. 2-14 are various embodiments of coiled tags, in accordance with some embodiments provided herein.

FIGS. 15-18 are various geometries of coiled tags, in accordance with some embodiments provided herein.

FIGS. 19A-19B show the movement of a hinged straight tag, in accordance with some embodiments provided herein.

FIGS. 20A-20C show a method of placing a stent through a coiled tag, in accordance with some embodiments provided herein.

Like reference numbers represent corresponding parts throughout.

### DETAILED DESCRIPTION

This document describes methods and materials for holding two anatomic structures in apposition. For example, this document describes methods and devices for holding a stomach and jejunum in apposition via a coiled tag.

A gastrojejunostomy is a surgical procedure that directly connects the stomach to the small intestine, specifically the jejunum. The jejunum is the second part of the small intestine, located after the duodenum. The stomach and jejunum can be connected by a small tube that allows food, liquids, and/or medications to bypass the duodenum. A gastrojejunostomy can be beneficial to individuals who cannot properly digest food, such as those who have stomach or duodenum blockages. These blockages can be caused by stomach or pancreatic cancer.

The coiled tags described herein can be implanted using a needle. Accordingly, the devices and methods provided herein can increase the safety and efficiency of connecting two anatomical structures. Further, the devices and methods can reduce the risk of the procedure failing due to anatomic structure movement.

The coiled tags devices can be composed of two coils with an intervening central segment connecting the coils. In some cases, the coiled tags can be entirely delivered via a hollow needle. Optionally, the device can be made from a substance that has shape memory and/or superelasticity, such as nitinol (e.g., 0.018 inches to 0.035 inches). The wire caliber can be such that the device can pass through the lumen of a needle. In some cases, the wire can be round, flat, or have another cross sectional shape. These properties allow the device to be straightened for delivery via a needle. When the device deploys from the tip of the needle, it assumes its preset configuration.

In some cases, coil tags will be delivered using endoscopic ultrasound (EUS). For example, to facilitate endoscopic gastrojejunostomy, an echoendoscope can be positioned in the stomach so that EUS views of an adjacent loop of jejunum are obtained. A needle can be passed via the echoendoscope and across the gastric and jejunal walls into the jejunal lumen. The coil tags can be pushed through the needle lumen until the first portion is in the jejunal lumen. The needle can then be pulled back into the stomach where the remaining portion of the device can be deployed. The coil tags can hold the jejunum against the stomach. In some cases, multiple (e.g., two to six) coil tags could be placed to secure the jejunum to stomach. In some cases, the coil tags could be placed in a circular shape to facilitate passage creation. Optionally, stomach-to-jejunal-lumen communication can be created using endoscopic devices and stents. In some cases, a large-diameter coil tag with a central opening can be used to provide circumferential apposition of a gastrojejunostomy site prior to creation of the gastrojejunostomy. Other potential uses for coil tags include apposition of the gallbladder to the stomach or duodenum, or apposition of the bile duct to the stomach or duodenum, etc. Coil tags may be curved to appose a curving luminal surface (e.g., a small bowel, a stomach, gallbladder, bile duct, pancreatic duct, trachea, bronchus, ureter, bladder, colon, heart chamber, blood vessel, etc.).

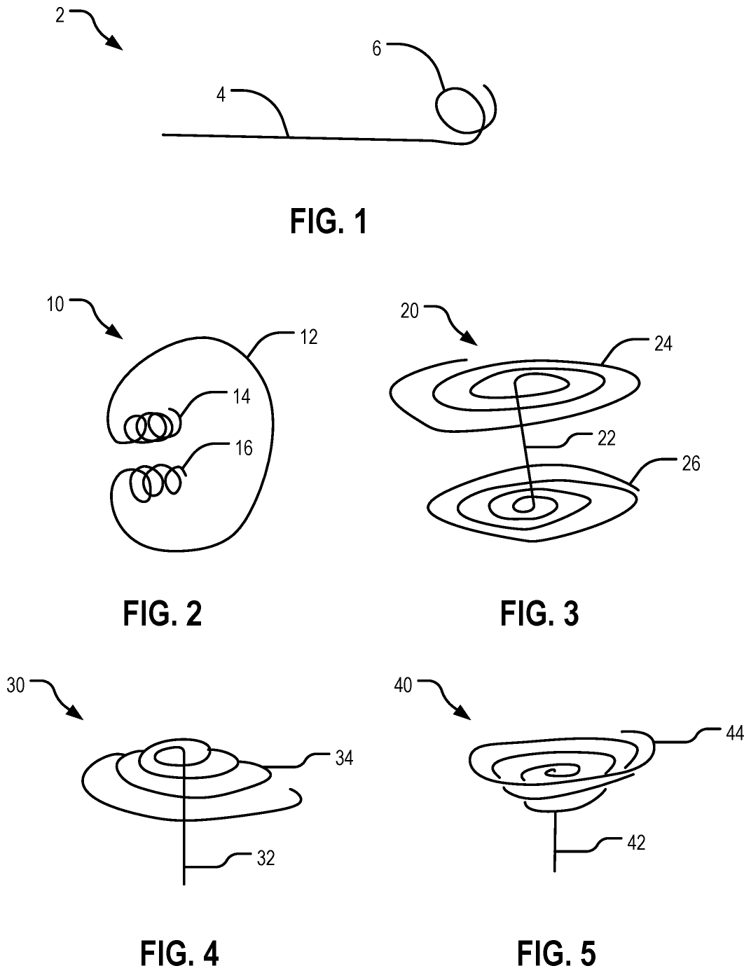

Referring to FIG. 1, a coiled tag 2 can include a straight portion 4 and a curved portion 6. The straight portion 4 can pass through two anatomic structures, while the curved portion 6 can remain in the cavities of the two anatomic structures to hold the structures in apposition. In this example, the curved portion 6 has no more than two 360-degree turns of the wire.

Referring to FIG. 2, a coiled tag 10 can include a connecting member 12, a first end coil 14, and a second end coil 16. Connecting member 12 can be generally C-shaped and configured to pass through, and remain in, two anatomic structures. The first end coil 14 and the second end coil 16 can be configured to remain in the cavities of the two anatomic structures to hold the structures in apposition. The first end coil 14 and the second end coil 16 can be substantially similar. In some cases, the first end coil 14 and the second end coil 16 can be mirror images of one another. The first end coil 14 and the second end coil 16 can extend longitudinally from the connecting member 12 toward a central portion of the connecting member 12. In some cases, the first end coil 14 and the second end coil 16 can have a helical shape.

Referring to FIG. 3, a coiled tag 20 can include a connecting member 22, a first end coil 24, and a second end coil 26. Connecting member 22 can be generally straight and configured to pass through, and remain in, two anatomic structures. The first end coil 24 and the second end coil 26 can be configured to remain in the cavities of the two anatomic structures to hold the structures in apposition. The first end coil 24 and the second end coil 26 can be substantially similar. In some cases, the first end coil 24 and the second end coil 26 can be mirror images of one another. The first end coil 24 and the second end coil 26 can extend substantially perpendicular from the connecting member 22 in a radially expanding configuration.

Referring to FIG. 4, a coiled tag 30 can include a connecting member 32, a first end coil 34, and a second end coil (not shown). Connecting member 32 can be generally straight and configured to pass through, and remain in, two anatomic structures. The first end coil 34 and the second end coil can be configured to remain in the cavities of the two anatomic structures to hold the structures in apposition. The first end coil 34 can extend substantially perpendicular from the connecting member 22 in a radially expanding configuration. The first end coil 34 can further extend toward connecting member 32, creating a conical shape with the tip pointing away from connecting member 32. The first end coil 34 and the second end coil can be substantially similar. In some cases, the first end coil 34 and the second end coil can be mirror images of one another.

Referring to FIG. 5, a coiled tag 40 can include a connecting member 42, a first end coil 44, and a second end coil (not shown). Connecting member 42 can be generally straight and configured to pass through, and remain in, two anatomic structures. The first end coil 44 and the second end coil can be configured to remain in the cavities of the two anatomic structures to hold the structures in apposition. The first end coil 44 can extend substantially perpendicular from the connecting member 42 in a radially expanding configuration. The first end coil 44 can further extend away from connecting member 42, creating a conical shape with the tip pointing toward connecting member 42. The first end coil 44 and the second end coil can be substantially similar. In some cases, the first end coil 44 and the second end coil can be mirror images of one another.

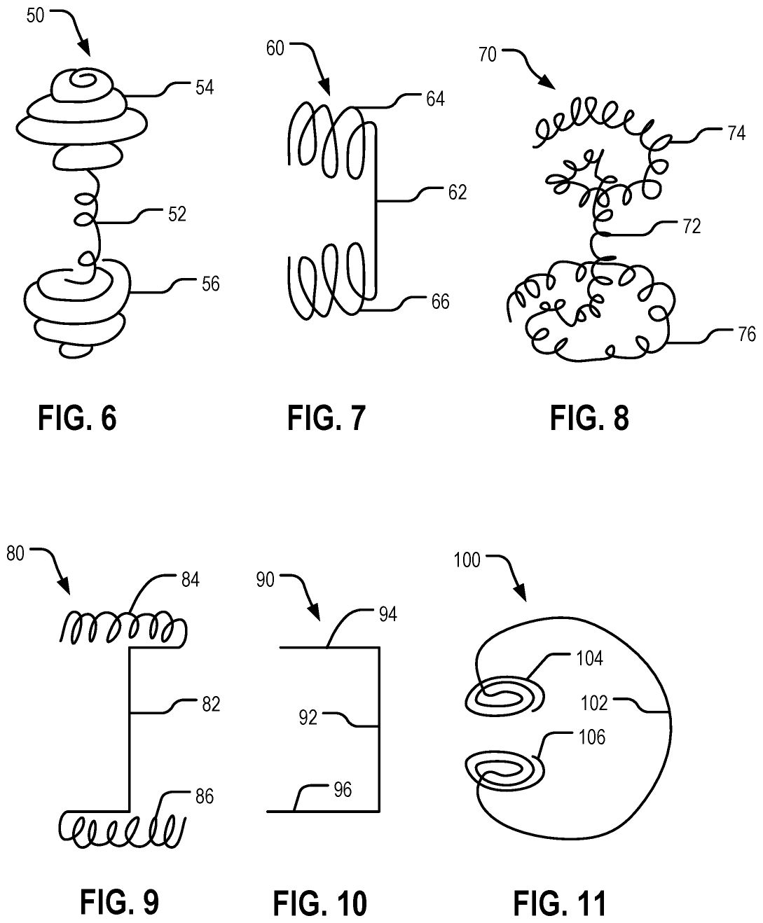

Referring to FIG. 6, a coiled tag 50 can include a connecting member 52, a first end coil 54, and a second end coil 56. Connecting member 52 can extend generally straight between the first end coil 54 and second end coil 56. The connecting member 52 can further be wound in a spiral configuration while extending between the first end coil 54 and the second end coil 56. Connecting member 52 can be configured to pass through, and remain in, two anatomic structures. The first end coil 54 and the second end coil 56 can be configured to remain in the cavities of the two anatomic structures to hold the structures in apposition. The first end coil 54 and the second end coil 56 can extend substantially perpendicular from the connecting member 52 in a radially expanding configuration. The first end coil 54 and the second end coil 56 can further extend toward connecting member 52, creating a conical shape with the tip pointing away from connecting member 52.

Referring to FIG. 7, a coiled tag 60 can include a connecting member 62, a first end coil 64, and a second end coil 66. Connecting member 62 can extend generally straight between the first end coil 64 and second end coil 66. Connecting member 62 can be configured to pass through, and remain in, two anatomic structures. The first end coil 64 and the second end coil 66 can be configured to remain in the cavities of the two anatomic structures to hold the structures in apposition. The first end coil 64 and the second end coil 66 can extend substantially perpendicular away from the connecting member 62. The first end coil 64 and the second end coil 66 can further extend away from the connecting member 62 in a spiral or helical configuration. In some cases, the first end coil 64 and the second end coil 66 can extend from the connecting member 62 in the same direction. In some cases, the first end coil 64 and the second end coil 66 can extend from the connecting member 62 in opposite directions.

Referring to FIG. 8, a coiled tag 70 can include a connecting member 72, a first end coil 74, and a second end coil 76. Connecting member 72 can extend generally straight between the first end coil 74 and second end coil 76. The connecting member 72 can further be wound in a spiral configuration while extending between the first end coil 74 and the second end coil 76. Connecting member 72 can be configured to pass through, and remain in, two anatomic structures. The first end coil 74 and the second end coil 76 can be configured to remain in the cavities of the two anatomic structures to hold the structures in apposition. The first end coil 74 and the second end coil 76 can extend substantially perpendicular from the connecting member 72 in a radially expanding configuration. In some cases, the first end coil 74 and the second end coil 76 have no more than one 360-degree turn of the wire. In some cases, the first end coil 74 and the second end coil 76 can further have a spiral configuration while radially expanding away from connecting member 72. The first end coil 74 and the second end coil 76 can be substantially similar. In some cases, the first end coil 74 and the second end coil 76 can be mirror images of one another.

Referring to FIG. 9, a coiled tag 80 can include a connecting member 82, a first end coil 84, and a second end coil 86. Connecting member 82 can extend generally straight between the first end coil 84 and second end coil 86. Connecting member 82 can be configured to pass through, and remain in, two anatomic structures. The first end coil 84 and the second end coil 86 can be configured to remain in the cavities of the two anatomic structures to hold the structures in apposition. The first end coil 84 and the second end coil 86 can extend substantially perpendicular away from the connecting member 82 in a first direction and can have a generally straight configuration. The first end coil 84 and the second end coil 86 can further extend away from the connecting member 82 in a second direction in a spiral or helical configuration, such that the first end coil 84 and the second end coil 86 include a straight portion and a spiral portion. In some cases, the first end coil 84 and the second end coil 86 can extend away from the connecting member 82 in the same direction for the first direction and the second direction. In some cases, the first end coil 84 and the second end coil 86 can extend away from the connecting member 82 in opposite directions for the first direction and the second direction.

Referring to FIG. 10, a coiled tag 90 can include a connecting member 92, a first end coil 94, and a second end coil 96. Connecting member 92 can extend generally straight between the first end coil 94 and second end coil 96. Connecting member 92 can be configured to pass through, and remain in, two anatomic structures. The first end coil 94 and the second end coil 96 can be configured to remain in the cavities of the two anatomic structures to hold the structures in apposition. The first end coil 94 and the second end coil 96 can extend substantially perpendicular away from the connecting member 92 in a first direction and can have a generally straight configuration. In some cases, the first end coil 94 and the second end coil 96 can extend away from the connecting member 92 in the same direction. In some cases, the first end coil 94 and the second end coil 96 can extend away from the connecting member 92 in opposite directions.

Referring to FIG. 11, a coiled tag 100 can include a connecting member 102, a first end coil 104, and a second end coil 106. Connecting member 102 can be generally C-shaped and configured to pass through, and remain in, two anatomic structures. The first end coil 104 and the second end coil 106 can be configured to remain in the cavities of the two anatomic structures to hold the structures in apposition. The ends of connecting member 102 can face one another such that the first end coil 104 and the second end coil 106 are in proximity to one another. The first end coil 104 and the second end coil 106 can extend substantially perpendicular from the connecting member 102 in a radially expanding configuration. In some cases, the first end coil 104 and the second end coil 106 can be substantially similar. In some cases, the first end coil 104 and the second end coil 106 can be mirror images of one another.

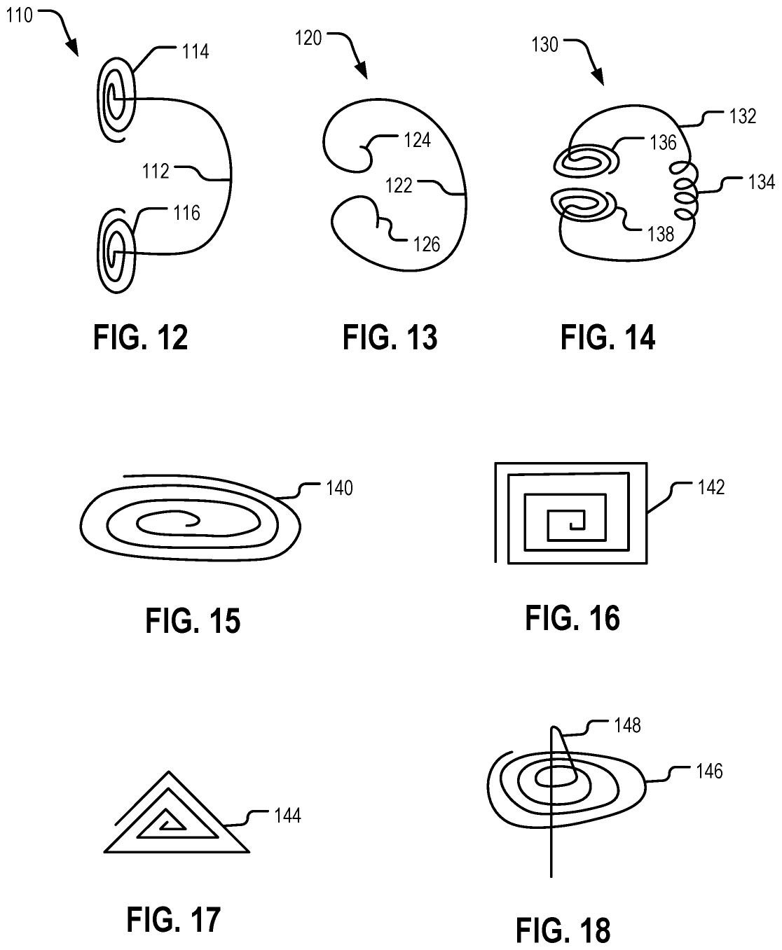

Referring to FIG. 12, a coiled tag 110 can include a connecting member 112, a first end coil 114, and a second end coil 116. Connecting member 112 can be generally C-shaped and configured to pass through, and remain in, two anatomic structures, with a central portion of the connecting member 112 remaining in a cavity of one of the anatomic structures. The first end coil 114 and the second end coil 116 can be configured to remain in a cavity of the other of the anatomic structures to hold the structures in apposition. The ends of connecting member 112 can extend substantially parallel from a central region of the connecting member 112. The first end coil 114 and the second end coil 116 can extend substantially perpendicular from the connecting member 112 in a radially expanding configuration. In some cases, the first end coil 114 and the second end coil 116 can be substantially similar. In some cases, the first end coil 114 and the second end coil 116 can be mirror images of one another.

Referring to FIG. 13, a coiled tag 120 can include a connecting member 122, a first end coil 124, and a second end coil 126. Connecting member 122 can be generally C-shaped and configured to pass through, and remain in, two anatomic structures, with a central portion of the connecting member 122 remaining in a cavity of one of the anatomic structures. The first end coil 124 and the second end coil 126 can be configured to remain in a cavity of the other of the anatomic structures to hold the structures in apposition. The ends of connecting member 122 can face one another such that the first end coil 124 and the second end coil 126 are in proximity to one another. In some cases, the first end coil 124 and the second end coil 126 have no more than one 360 degree turn of the wire. In some cases, the first end coil 124 and the second end coil 126 can be substantially similar. In some cases, the first end coil 124 and the second end coil 126 can be mirror images of one another.

Referring to FIG. 14, a coiled tag 130 can include a connecting member 132, a first end coil 136, and a second end coil 138. Connecting member 132 can be generally C-shaped and configured to pass through, and remain in, two anatomic structures. Connecting member 132 can also include a spiral region 134. Spiral region 134 can be configured to remain in the soft tissue or walls of the two anatomic structures to limit movement of the coiled tag 130. In some cases, the spiral region 134 can be embedded in a lumen. In some cases, coiled tag 130 can be permanent. In some cases, spiral region 134 can be located at a central location of connecting member 132. In some cases, spiral region 134 can be located along a different portion of connecting member 132. The first end coil 136 and the second end coil 138 can be configured to remain in the cavities of the two anatomic structures to hold the structures in apposition. The ends of connecting member 132 can face one another such that the first end coil 136 and the second end coil 138 are in proximity to one another. The first end coil 136 and the second end coil 138 can extend substantially perpendicular from the connecting member 132 in a radially expanding configuration. In some cases, the first end coil 136 and the second end coil 138 can be substantially similar. In some cases, the first end coil 136 and the second end coil 138 can be mirror images of one another.

Any of the coiled tags of FIGS. 1-14 can include a first end coil and/or a second end coil of a different geometry, such as the geometries shown in FIGS. 15-18.

Referring to FIG. 15, an end coil 140 can have a generally radially expanding shape. For example, the end coil 140 can have a round spiral shape. In some cases, the end coil 140 can be similar to a circle, oval, or ellipse in shape.

Referring to FIG. 16, an end coil 142 can have a generally radially expanding shape. For example, the end coil 142 can have a quadrilateral spiral shape. In some cases, the end coil 142 can be similar to a square, rectangle, parallelogram, diamond, or rhombus in shape.

Referring to FIG. 17, an end coil 144 can have a generally radially expanding shape. For example, the end coil 144 can have a triangular spiral shape. In some cases, the end coil 144 can be similar to a right triangle, isosceles triangle, obtuse triangle, or diamond in shape.

Similarly, an end coil can have a shape similar to a pentagon, hexagon, heptagon, octagon, and so on. In some cases, the end coil can have a shape that extends in 3 dimensions, creating a conical shape or a pyramid with a base of three or more sides.

Referring to FIG. 18, an end coil 146 can have a generally radially expanding shape. For example, the end coil 146 can have a shape as described with respect to FIGS. 15-17. In some cases, the end coil 146 can further include a distal protrusion 148. The distal protrusion 148 can protrude beyond the end coil 146. In some cases, the distal protrusion is part of a central segment joining two end coils. Optionally, the distal protrusion 148 can aid in facilitating removal of the coiled tag.

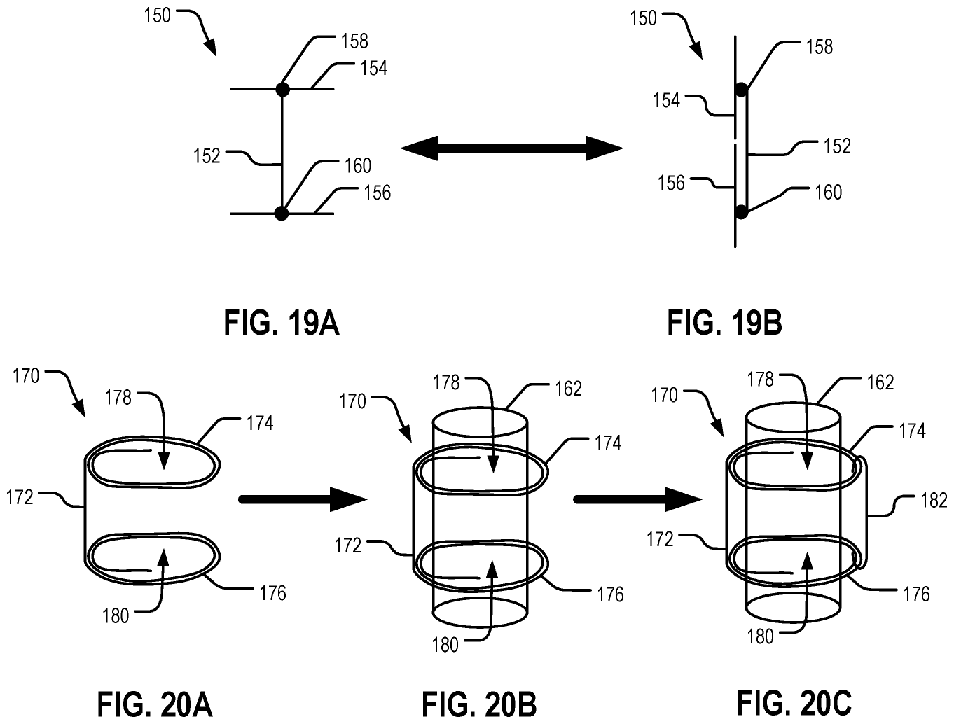

Referring to FIGS. 19A-19B, a coiled tag 150 can include a connecting member 152, a first end coil 154, and a second end coil 156. Connecting member 152 can extend generally straight between the first end coil 154 and second end coil 156. Connecting member 152 can be configured to remain in a cavity of one of the anatomic structures. The first end coil 154 and the second end coil 156 can be configured to remain in a cavity of the other of the anatomic structures to hold the structures in apposition. The first end coil 154 and the second end coil 156 can extend substantially perpendicular away from the connecting member 152 in a first direction and a second direction. The first end coil 154 and the second end coil 156 can have a generally straight configuration.

The coiled tag 150 can further include a first hinge 158 and a second hinge 160. The first hinge 158 can couple the connecting member 152 and the first end coil 154, and the second hinge 160 can couple the connecting member 152 and the second end coil 156. Accordingly, the coiled tag 150 can move from a first position (FIG. 19A) where the first end coil 154 and the second end coil 156 are generally perpendicular to the connecting member 152, to a second position (FIG. 19B) where the first end coil 154 and the second end coil 156 are generally parallel to the connecting member 152. Accordingly, coiled tag 150 can be delivered through a needle with a narrow profile, as seen in FIG. 19B, and then be implanted in a configuration, as seen in FIG. 19A, which enables two anatomic structures to be held in apposition.

Referring to FIGS. 20A-20C, a method of placing a stent 162 through a coiled tag 170 is shown. The coiled tag 170 can include a connecting member 172, a first end coil 174, and a second end coil 176. Connecting member 172 can extend generally straight between the first end coil 174 and second end coil 176. Connecting member 172 can be configured to pass through, and remain in, two anatomic structures. The first end coil 174 and the second end coil 176 can be configured to remain in the cavities of the two anatomic structures to hold the structures in apposition. The first end coil 174 and the second end coil 176 can extend substantially perpendicular away from the connecting member 172. The first end coil 174 and the second end coil 176 can further extend away from the connecting member 172 and extend back toward the connecting member 172, creating a first loop 178 and a second loop 180. In some cases, the loop 178 can include multiple 360-degree turns. The first end coil 174 and the second end coil 176 can extend from the connecting member 62 in the same direction, such that the loops 178 and 180 align. As shown in FIG. 20B, once coiled tag 170 is implanted, the stent 162 can be implanted through loops 178 and 180. In some cases, a different lumen-creating device can be used to create an opening within loops 178 and 180. As shown in FIG. 20C, coiled tag 170 can further include a second connecting member 182. Second connecting member 182 can be introduced via a needle and connect the first end coil 174 and a second end coil 176, opposite connecting member 172. In some cases, second connecting member 182 can be attached to the first end coil 174 and the second end coil 176 after introducing stent 162. In some cases, second connecting member 182 can be attached to the first end coil 174 and the second end coil 176 before introducing stent 162.

In some cases, the diameter of coils and length of the central segment can vary by application. Optionally, the distal coil and the central segment may have a different color or etching to facilitate identification of the central segment during deployment.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments. It should also be understood that the described program components and systems can generally be integrated together in a single product or packaged into multiple products.

Particular embodiments of the subject matter have been described. Other embodiments are within the scope of the following claims. For example, the actions described in the claims can be performed in a different order and still achieve desirable results. As one example, the process depicted in the accompanying figures do not necessarily require the particular order shown, or sequential order, to achieve desirable results. In certain implementations, multitasking and parallel processing may be advantageous.

1. A device for holding two anatomical structures in apposition, the device comprising:
a first holding member;
a second holding member; and
a connecting member extending between the first holding member and the second holding member.

2. The device of claim 1, wherein the device is made of shape memory material.

3. The device of claim 1, wherein the connecting member is substantially straight extending between the first holding member and the second holding member.

4. The device of claim 1, wherein the connecting member is curved such that the first holding member and the second holding member are in proximity to one another, or wherein the connecting member is non-linear.

5. (canceled)

6. The device of claim 1, wherein the connecting member is configured to anchor the device in a tissue.

7. The device of claim 1, wherein the first holding member and the second holding member extend perpendicular from the connecting member in a radially expanding spiral pattern.

8. The device of claim 1, wherein the first holding member and the second holding member extend longitudinally from the connecting member in a helical configuration.

9. The device of claim 1, wherein the first holding member and the second holding member extend from the connecting member in a radially expanding conical configuration, or wherein the first holding member and the second holding member extend toward the connecting member in a radially expanding conical configuration.

10. (canceled)

11. The device of claim 1, wherein the connecting member has a helical configuration or includes a helical portion.

12. (canceled)

13. The device of claim 1, wherein the first holding member includes a distal protrusion.

14. The device of claim 1, further comprising a hinge coupling the first holding member and the connecting member.

15. The device of claim 1, wherein the first holding member is configured to be positioned in a first anatomical structure, wherein the second holding member is configured to be positioned in the first anatomical structure, and wherein the connecting member is configured to extend through the first anatomical structure and a second anatomical structure.

16. The device of claim 1, wherein the first holding member is configured to be positioned in a first anatomical structure, wherein the second holding member is configured to be position in a second anatomical structure, and wherein the connecting member is configured to extend through the first anatomical structure and the second anatomical structure.

17. The device of claim 16, wherein the first holding member is configured to allow passage of a stent through a portion of the first holding member, or wherein the second holding member is configured to allow passage of a stent through a portion of the second holding member.

18. (canceled)

19. The device of claim 16, further comprising a second connecting member configured to extend between the first holding member and the second holding member opposite the connecting member.

20. A method of holding two anatomical structures in apposition, the method comprising:
implanting a device in the two anatomical structures by:
ejecting a first holding member from a needle in a first anatomical structure;
withdrawing the needle into a second anatomical structure; and
ejecting a second holding member from the needle in the second anatomical structure.

21. The method of claim 20, wherein withdrawing the needle further comprises ejecting a connecting member in the first anatomical structure and the second anatomical structure, wherein the connecting member extends between the first holding member and the second holding member.

22. The method of claim 20, further comprising implanting a plurality of devices, wherein the plurality of devices comprises two to six devices.

23. (canceled)

24. The method of claim 22, further comprising implanting a stent, wherein implanting the stent comprises implanting the stent in a first loop of the first holding member or in a second loop of the second holding member.

25-26. (canceled)

27. The method of claim 24, wherein implanting the stent comprises implanting the stent between the plurality of devices.
